(19) United States
(12) Patent Application Publication
(10) Pub. No.: US 2024/0299770 A1
(43) Pub. Date: Sep. 12, 2024

(54) CRANIAL DETOXIFIER DEVICE

(71) Applicants: Ling-Ho LAM, San Jose, CA (US); Raymond Yim CHIU, San Jose, CA (US)

(72) Inventors: Ling-Ho LAM, San Jose, CA (US); Raymond Yim CHIU, San Jose, CA (US)

(21) Appl. No.: 18/485,389

(22) Filed: Oct. 12, 2023

(51) Int. Cl. A61N 5/06; A61H 39/04; A61H 39/06

(52) U.S. Cl. CPC A61N 5/0619; A61H 39/04; A61H 39/06; A61H 2039/005; A61H 2201/10; A61H 2201/169; A61N 2005/0626; A61N 2005/0651; A61N 2005/0659; A61N 2005/0663

(57) ABSTRACT

A cranial detoxifier device (CDD) that includes a free electron generator module, an infrared (IR) generator module, a heater module, a visible light generator, and a microcontroller that controls the free electron generator module, the infrared generator module, the heater module, and the visible light generator, the microcontroller and components of the free electron generator module, the IR generator module, the heater module, and the visible light generator residing in the housing of the CDD.

### FIELD OF THE INVENTION

The present invention relates to a detoxifier device, and more particularly to a cranial detoxifier device (CDD).

### BACKGROUND OF THE INVENTION

Dementia is not a specific disease but is rather a general term for the impaired ability to remember, think, or make decisions that interferes with doing everyday activities. Alzheimer's disease is the most common type of dementia. Though dementia mostly affects older adults, it is not a part of normal aging.

According to the World Alzheimer Report 2023, the World Health Organization expects the number of people living with dementia across the world to rise from 55 million in 2019 to 139 million in 2050. The costs associated with dementia are also expected to more than double from US$1.3 trillion per year in 2019 to $2.8 trillion dollars by 2030.

People with dementia have problems with memory, attention, communication, reasoning, judgment, problem solving, and visual perception beyond typical age-related changes in vision.

Signs that may point to dementia include getting lost in a familiar neighborhood, using unusual words to refer to familiar objects, forgetting the name of a close family member or friend, forgetting old memories, and not being able to complete tasks independently.

Treatments of dementia include pharmacological interventions and non-pharmacological interventions.

It has been alleged that treatment in the early stages slows down degeneration.

Since the 1980s, it has been commonly believed that Alzheimer's disease is caused by the accumulation in the brain of sticky synapse-destroying plaques made of a piece of a protein called amyloid-beta.

Lab studies indicate that amyloid-beta is formed in the brain by a series of steps, and that either intervening to interrupt those steps or destroying amyloid-beta plaques would be an effective way to treat and even prevent Alzheimer's disease.

Alzheimer's disease is not a single disease although the symptoms might make it look like it is. Rather than being caused by the buildup of sticky amyloid-beta plaques, Alzheimer's is actually the result of a protective response in the brain.

There are three main subtypes of Alzheimer's disease: 1) inflammation (from infection, diet, or other causes); 2) decline and shortage of supportive nutrients, hormones, and other brain-supporting molecules; 3) toxic substances such as metals or biotoxins (poisons produced by microbes such as molds). Each subtype is driven by different biochemical processes.

Chronic inflammation is often caused by frequent and persistent acute inflammation, as well as viral infections, autoimmune reactions, and foreign bodies that are not removed from inside the brain.

Red light therapy (650 nm red light wavelength) alleviates chronic inflammation by increasing blood flow to the damaged tissues, and has been found in numerous clinical trials to increase the body's antioxidant defenses.

Dr. Michael Hamblin of Harvard Medical School and Massachusetts General Hospital is one of the world's leading photomedicine researchers on Joovv's Scientific Advisory Board. He has studied light therapy at length and concluded that one of its most reproducible effects is an overall reduction in inflammation, which is particularly important for disorders of joints, traumatic injuries, and lung and brain disorders. Dr. Hamblin has explained that wavelengths of natural red and near infrared (IR) light are “a very mild form of stress that activates protective mechanisms in the cells”. For example, when longer wavelengths or visible “red light hits the skin, it nudges mitochondria to make energy more efficiently and boost production of healing anti-inflammatories or disease-fighting antioxidants.”

The researchers led by Dr. Stuart Peirson from Oxford's Sleep and Circadian Neuroscience Institute, Oxford University, have discovered the effects of light emitting devices on sleep. For example, recent studies have shown that blue light delays sleep.

An NIH-funded study has suggested that sleep clears the brain of damaging molecules associated with neurodegeneration. Green light with a wavelength of 525 nm may achieve the desired effect by inducing sleep. On the other hand, blue light having a wavelength of 380 nm has been shown to reduce inflammation and brain cell recovery.

It is well known that heat affects the dynamics of all physicochemical processes governing neural activity. It is also known that the brain has high levels of metabolic activity, and all energy used for brain metabolism is finally transformed into heat. However, the issue of brain temperature as a factor reflecting neural activity and affecting various neural functions remains in the shadow and is usually ignored by most physiologists and neuroscientists.

Moxibustion (heat induction into body) is a Traditional Chinese Medicine technique that involves burning of mugwort (Artemisia Vulgaris), a small spongy herb, on particular acupoints on the body to facilitate healing. Moxibustion “heat” therapy mainly helps to warm the meridians, improve blood circulation, dispel cold and dampness, and strengthen the immune system. Scientific reviews have reported an increase in red blood cells, white blood cells, and platelets around the area of treatment, effectively improving oxygen uptake by the cells of the body and driving the targeted systemic functions. Many patients report a warm, soothing sensation during and after a session of moxibustion.

In recent years nanotechnology development has provided functional sportswear with many properties to enhance sports activities, performance, efficiency and comfort. For example, sportswear should allow the wearer to stay warm in a cold environment, and keep cool in hot situations through transferring sweat away from the skin. In general, the operating mechanism of IR radiating materials is to transform heat energy generated by the body (convection and conduction) into radiation within the IR wavelength range (between 3˜20 μm) to induce homeostasis and photobiomodulation through deeper penetration of IR radiation and water molecule absorption in the skin. The use of materials that generate IR radiation is possibly helpful to enhance blood circulation and the metabolism of human body.

Low-level light therapy (LLLT) is defined as treatment using irradiation with light of low power intensity of a particular frequency, for example, IR. Research has shown that IR radiation may induce promotion of cell viability and growth factors, which elicit potential therapeutic effects on the brain injury or degenerative brain disease. Individuals with brain disorders including TBI, Alzheimer's disease, Parkinson's disease, and stroke could benefit from IR induced ATP synthesis, growth factor production, anti-inflammatory effects and anti-apoptosis.

It is well established, though not widely known, that the surface of the earth possesses a limitless and continuously renewed supply of free or mobile electrons as a consequence of a global atmospheric electron circuit. Wearing shoes with insulating soles and/or sleeping in beds that are isolated from the electrical ground plane of the earth have disconnected most people from the earth's electrical rhythms and free electrons. The most reasonable hypothesis to explain the beneficial effects of earthing is that a direct earth connection enables both diurnal electrical rhythms and free electrons to flow from the earth to the body. It has been proposed that the earth's diurnal electrical rhythms set the biological clocks for hormones that regulate sleep and activity. It has also been suggested that free electrons from the earth neutralize the positively charged free radicals that are the hallmark of chronic inflammation.

A cranial detoxifier device (CDD), according to the present invention includes a free electron generator module; an infrared (IR) generator module; a heater module; a visible light generator; and a microcontroller that controls the free electron generator module, the infrared generator module, the heater module, and the visible light generator.

The CDD may further include a housing that houses the microcontroller and components of the free electron generator module, the IR generator module, the heater module, and the visible light generator.

The CDD according to the present invention further includes a first metallic stud and a second metallic stud extending out of a head section of the housing.

Each metallic stud may have a dome-shaped end.

The first and second metallic studs may serve as output components of the free electron generator.

The first and second metallic studs may transmit heat from the output components of the heater module

The first and second metallic studs may transmit heat from the output components of the heater module in the temperature range of 35°-65° C.

Each metallic stud may contain gemstones in powder form.

An LED component may serve as the output component of the visible light generator.

An orifice may be defined in the head section with the LED component aligned with the orifice to transmit light through the head section.

The LED component may emit one of red light, green light, and blue light, or may emit red light, green light, and blue light.

The output component of the IR generator module may be an infrared generating coil residing within a head section of the housing.

The IR generating coil and the LED component may be operable in the range 5-50 Hz.

The first and second metallic studs may serve as a charge head and a discharge head of the free electron generator module, wherein, in operation, the charge head is at a voltage in the range 20-30 volts relative to the discharge head and the free electron generator module generates less than 0.03 amps.

The CDD according to the present invention may further include a container containing gemstones in powder form.

The gemstones contained in the container or the metallic may include pure and clear white quartz, amethyst, clear aquamarine, rose quartz, clear citrine, Tiger's Eye, and Tektite.

The particles of the gemstones in the powder form are 0.84 mm or smaller.

The output of the free electron generator module, the IR generator module, the heater module, and the visible light generator may be adjustable in response to user instructions.

The user instructions may be received from a user-operable mobile device.

A system according to the present invention includes two or more CDDs and a carrier 20 in which each CDD has first and second metallic studs extending from a head section of the housing thereof, and each housing of each CDD has exterior threads that engage a threaded inner surface of a respective opening in the carrier whereby rotation of each CDD housing causes repositioning of the first and second metallic studs thereof relative to the carrier.

### SUMMARY OF THE INVENTION

It is an object of the present invention to provide a device that can interrupt the processes that cause dementia.

Toxin accumulation inside the cranial structure results in inflammation, calcification, and fibrosis of brain cells and neural networks that impede the cranial rhythm to toxin removal (amyloid-beta protein, dead brain cells, etc.).

A device according to the present invention generates free electrons to neutralize the positively charged free radicals leading to chronic inflammation inside the brain.

A device according to the present invention generates IR to enhance cranial blood circulation and boost the production of healing anti-inflammatories or disease-fighting antioxidants to cure damaged brain cells and neural networks.

A device according to the present invention generates low level light for low-level light therapy (LLLT) with body-generated IR signals to induce and promote cell viability and growth factors, which elicit potential therapeutic effects on a brain injury or a degenerative brain disease. Individuals suffering from TBI, Alzheimer's disease, Parkinson's disease, and stroke-related symptoms could benefit from IR-induced ATP synthesis, growth factor production, anti-inflammatory effects and anti-apoptosis induced promotion of cell viability and growth factors.

A device according to the present invention generates visible light to improve sleep quality and hormonal balance inside the brain while sleeping.

A device according to the present invention produces heat (moxibustion induction) to stimulate all acupressure points on the scalp, resulting in boosting blood circulation, toxin removal from the brain, enhancing self-healing capability in the chronic inflammation inside the brain.

A device according to the present invention is a CDD that can be used by patients or caretakers.

A CDD according to the present invention can be used to gently massage the scalp to relieve stress and clear blockages of soft tissues and/or blood vessels around the sutures of the skull, resulting in a sense of deep relaxation.

Specifically, a CDD according to the present invention has two metal stubs each with a dome-shaped head. The two metal studs are controlled by a circuitry to release heat energy at a user-defined level to simulate the effect of moxibustion. Through rubbing/massaging the scalp by the metal round heads, about 90 acupressure points located on the scalp can be stimulated which brings out the effect of containing/reversing brain cells/neural network degeneration, toxin removal inside the brain and introduction of nutrients to the brain through enhancing the cranial blood vessels circulations by relieving compression in the head, neck, and back. This can soothe pain and release both emotional and physical stress and tension. It is also thought to help restore cranial mobility and ease or release restrictions of the head, neck, and nerves.

A CDD according to the present invention has gemstones (in powder form) inside the two metal stubs to absorb the IR emitted by the brain continuously. The gemstones, upon receiving sufficient threshold energy level from IR generated by the body, will emit their own IR to bring about the effects of LLLT.

A CDD according to the present invention may enhance the removal of brain toxin, speed up anti-inflammation inside the brain, and improve cranial blood circulation, in order to achieve the reversal of degeneration of brain cells and neural networks inside the brain.

Other features and advantages of the present invention will become apparent from the following description of the invention which refers to the accompanying drawings.

### BRIEF DESCRIPTION OF THE DRAWINGS

FIG. 1 schematically shows the electronic components of a CDD according to the present invention.

FIG. 2A shows a top view of a first physical embodiment of a CDD according to the present invention.

FIG. 2B shows a front view of the CDD shown in FIG. 2A.

FIG. 2C shows a bottom perspective view of the CDD of FIGS. 2A and 2B.

FIG. 2D shows a top perspective view of the CDD of FIGS. 2A and 2B.

FIG. 3A shows two CDDs according to a second physical embodiment of the present invention assembled to a carrier at the beginning stage of assembly (the left CDD) and at the end of assembly (the right CDD).

FIG. 3B is the rear view of the assembly seen in FIG. 3A.

FIG. 4A shows an exploded view of the CDD according to the second embodiment.

FIG. 4B shows another exploded view of the CDD according to the second embodiment.

FIG. 4C shows a third exploded view of the CDD according to the second embodiment.

FIG. 4D shows a view of the CDD according to the second embodiment with a portion of the main body thereof removed from view to show the circuit board.

### DETAILED DESCRIPTION OF EMBODIMENTS OF THE INVENTION

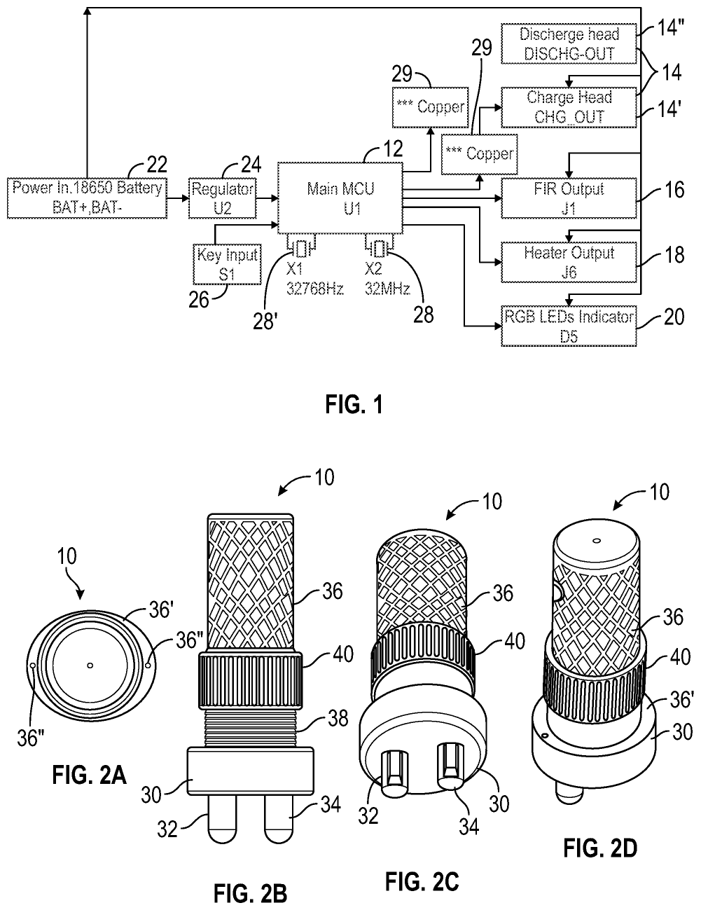

Referring to FIG. 1, a CDD 10 (see FIGS. 2A-2D, 3A-3B, 4A-4D) according the present invention includes a microcontroller unit (MCU) 12, which controls the operation of a free electron generator module 14, an infrared generator (IR generator) module 16, a heater module 18, and a visible light generator 20 all of which receive power from, preferably, a battery 22, which may be, but does not have to be, a rechargeable battery. Instead of a battery, another source of electrical power (for example, an electrical converter that is connectable to the power line) could be used without deviating from the present invention.

Preferably, a regulator 24 regulates the power from the battery 22 to the MCU 12, and a manual switch 26 turns the CDD ON and OFF. Other switches could be used without deviating from the present invention. For example, a wireless switch connectable to a wireless device such as a mobile smart phone or a tablet could be used to turn the MCU 12 ON or OFF.

Components 28, 28′ provide two clock frequencies for the MCU 12 for its operation.

The free electron generator module 14 is a DC power generator circuit that includes a charge head 14′ and a discharge head 14″ each connected to the MCU 12 via a respective nano copper wire 29. The charge head 14′ and the discharge head 14″ may be spaced electrodes. Current flows from the charge head 14′ to the discharge head 14″ when the charge head 14′ and the discharge head 14″ make direct contact with the user's skin. The charge head 14′ may be at 20-30V DC relative to the discharge head 14″ when the free electron generator module 14 is in operation. The free electron generator module 14 generates less than 0.03 Amps when operating. The gemstone powder filled metallic studs 32, 34 discussed below function as the charge head 14′ and discharge head 14″ electrodes of the free electron generator module 14.

In the preferred embodiment, the battery 22, the regulator 24, the manual switch 26, and components 28, 28′ are co-packaged with the MCU 12 and the other components of the modules in one common, portable housing as further described below.

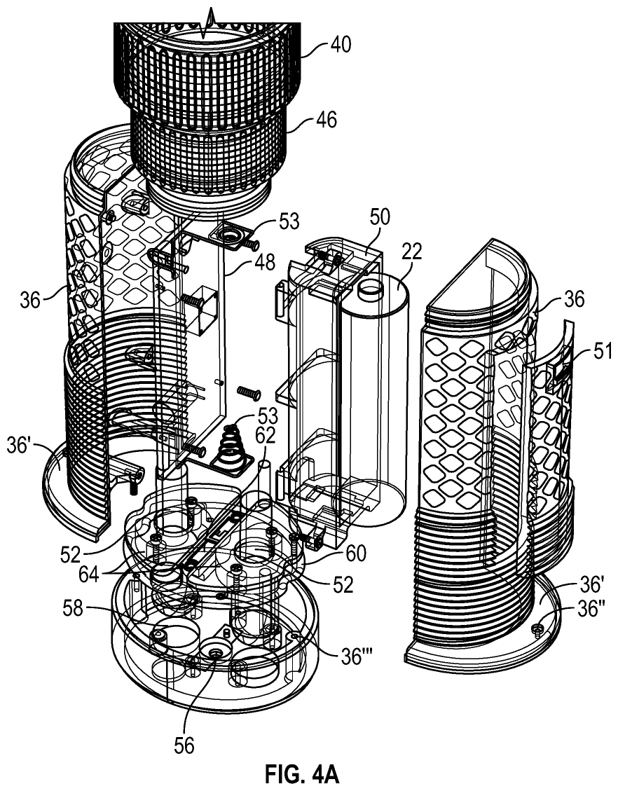

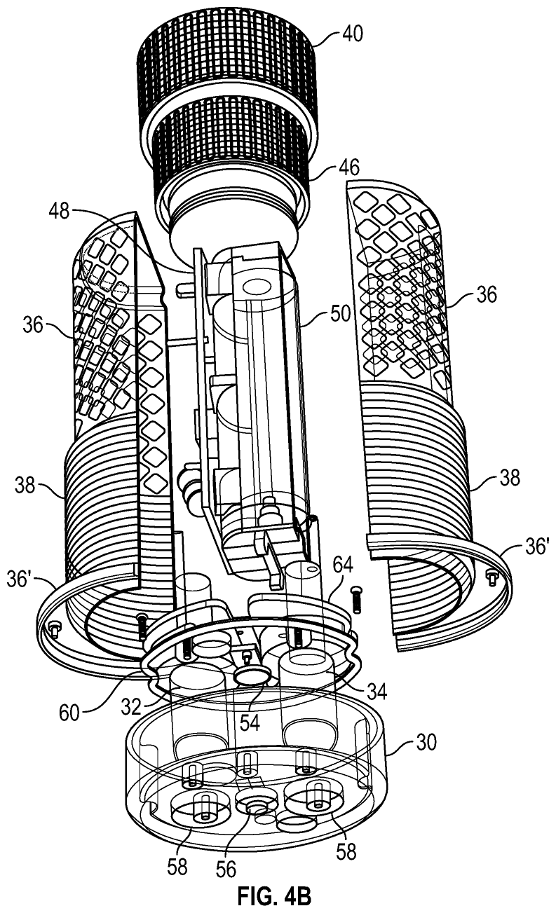

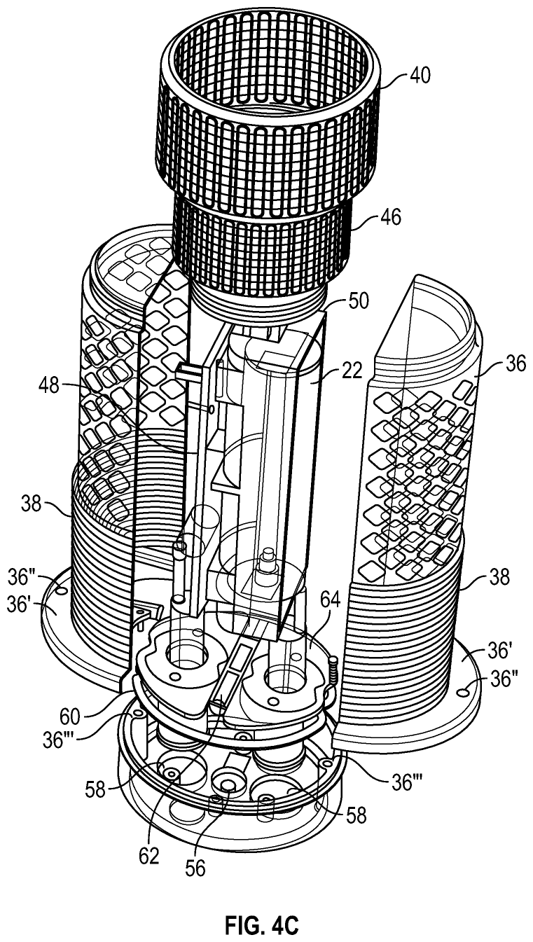

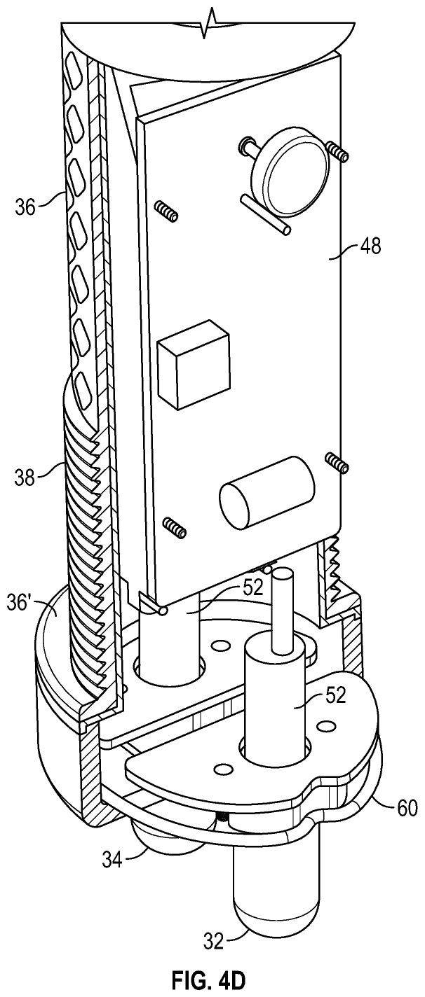

Referring to FIGS. 2A-2D, a CDD 10 according to an embodiment of the present invention includes a housing having a head section 30. Two metallic studs 32, 34 extend out of the head section 30. The head section 30 of the housing may be coupled to the main body 36 of the housing with, for example, screws or the like fasteners so that it may be removed from the main body 36. Specifically, the main body 36 may be a cylindrical body with a flanged part 36′ at an end thereof, which has screw holes 36″ that can receive screws which are then received in screw receptacles 36″′ defined in the head section 30 (FIG. 4C). The main body 36 includes exterior threads 38 that starts at a location near the head section 30 and terminates before reaching the end of the main body 36. In this embodiment, a nut 40 with interior threads may be provided to threadably engage the exterior threads 38.

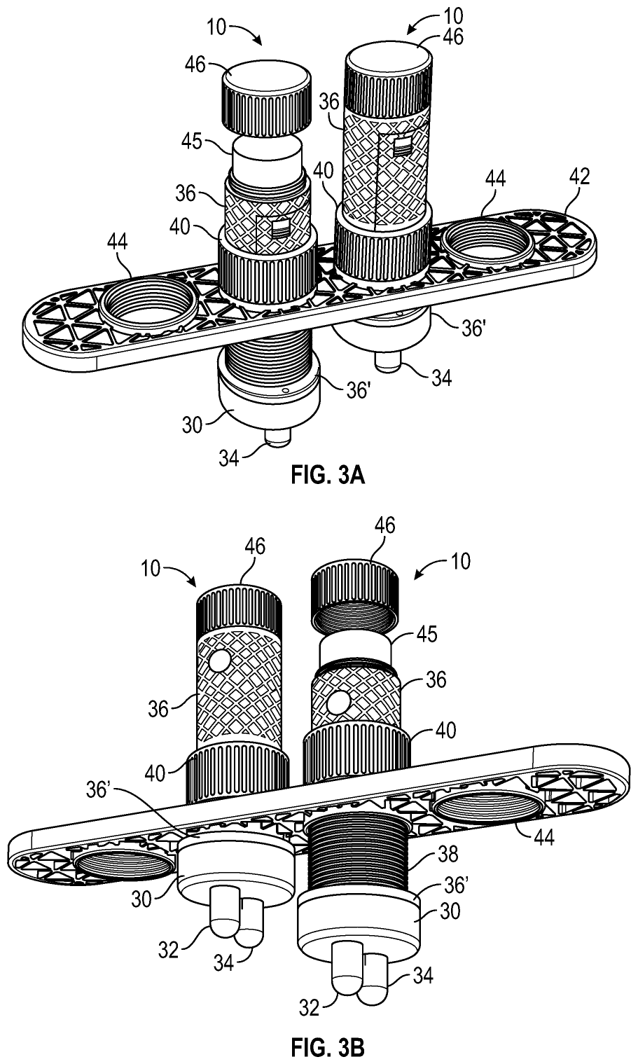

FIGS. 3A and 3B show two CDDs 10 and a carrier 42. The exterior threads 38 enables the main body 36 to be coupled to the carrier 42. Specifically, the carrier 42 may have openings 44 therein each with a threaded inner surface to engage the exterior threads 38 of the main body 36 of a CDD 10. The nut 40 when provided can be used to further secure the main body 36 to the carrier 42. Specifically, the flanged part 36′ of a CDD 10 is wider than an opening 44 in which the main body 36 is received. Once the main body 36 is fully received in an opening 44 (e.g. the CDD on the right in FIG. 3A and on the left in FIG. 3B) such that the flanged part 36′ is in contact with the underside of the carrier 42, the nut 40 can be rotated toward the carrier 42 until it makes contact with the top side of the carrier 42 whereby the carrier 42 is sandwiched between the flanged part 36′ and the nut 40 to further secure the main body 36 to the carrier 42.

The carrier 42 may be used by a user to press the studs 32, 34 of multiple CDDs 10 against the scalp. Exterior threads 38 allow for the adjustment of the position of the studs 32 and 34 relative to the carrier 42 when a CDD 10 is received in an opening 44. For example, when two to four CDDs 10 are received in the openings 44 in the carrier 42, by rotating the CDDs 10 in the openings 44 clockwise (causing inward movement with the studs 32, 34 moving toward the carrier 42, for example) or anti-clockwise (causing outward movement with the studs 32, 34 moving away from the carrier 42, for example) the relative distance between the studs 32, 34 of the CDDs 10 and the carrier 42 may be adjusted. Thereafter, a nut 40 may be used to lock each CDD 10 in place. By allowing the adjustment of the position of the studs 32, 34 relative to the carrier 42, the studs 32, 34 of the CDDs 10 may be positioned to touch the scalp simultaneously regardless of the cranial condition of the client (concave and convex irregularities on the scalp).

In the embodiments shown in FIGS. 3A and 3B, the main body 36 is provided with a removable cap 46 which is threadably coupled to the end of the main body 36 opposite the end that is coupled to the head section 30 to provide access to a (preferably cylindrical) container 45 that contains natural gemstones. Each gemstone may be provided for its ability to absorb energy from a respective chakra. In eastern medicine, chakras are considered the body's energy centers. There are seven chakras, namely: Root Chakra, Sacral Chakra, Solar Plexus Chakra, Heart Chakra, Throat Chakra, Third-Eye Chakra, and Crown Chakra, located along the spine and correspond to different physical and emotional functions. The chakras are believed to be pools or swirling disks of energy that bathe and fuel the organs in their proximity. It is believed that chakras govern the endocrine system, carry information about the person's history, and encode and process physical, mental, emotional, and spiritual experiences. The chakras play an essential role in the body's overall wellbeing, as any blockages or imbalances in these energy centers can manifest as physical, emotional, or mental issues. Chakra healing balances and unblocks these energy centers to promote optimal health and well-being.

The meridian theory was originally expressed by the Chinese on the basis of observations of illnesses and holistic treatment. In the meridian theory, chakras deliver their energy to the organs. It is believed that as the body's energy bloodstream, the meridian system brings vitality and balance, removes blockages, adjusts metabolism, and even determines the speed and form of cellular change. The flow of the meridian energy pathways is believed to be as critical as the flow of blood. It is believed that meridians affect every organ and every physiological system, including the immune system, the nervous system, the endocrine system, the circulatory system, the respiratory system, the digestive system, the skeletal system, the muscular system, and the lymphatic system. It is believed that each meridian network operates at a particular frequency in harmony with the other meridian networks, and that any deviation of one or more meridian networks may distort the body energy flow (especially in the brain) and progressively lead to chronic illnesses, including dementia.

The natural gemstones may include a combination of gemstones in powder form such as a combination of high quality, pure, and clear white quartz (for Crown Chakra), high quality amethyst (for Third-Eye Chakra), high quality clear aquamarine (for Throat Chakra), high quality rose quartz (for Heart Chakra), clear citrine (for Solar Plexus Chakra), Tiger's Eye (for Sacral Chakra), black obsidian and Tektite (for Root Chakra). The gemstone particles are intended to absorb IR released from the human body, store the IR energy until a threshold level is reached, and release the stored natural IR once the threshold is reached to stimulate the energy centers and bring about a recharge effect of the weak body energy center that contributed to the brain degeneration/inflammation, the blockage of the drainage system inside the brain, and the accumulation of amyloid-beta leading to the adverse influence of the normal functioning of brain cells and neural networks. The container 45 will reside at the closed end of the main body 36. The gemstone particles inside the container 45 are intended to release IR back to the surface of the body of the user, including the acupressure points on the body through which the corresponding meridian networks may be activated to improve the energy flow inside the body and ultimately enhance the self-healing capability of the body. Thus, the container 45 and the cap 46 are transparent to the IR received from the user's body and also transparent to the IR transmitted to the user's body from the gemstone material. The container 45 is a passive feature meaning it is not driven or controlled electronically. Furthermore, while the cap 46 may be held in contact with the human body so that the container is located as close as possible to the human body, physical contact is not necessary because IR radiating from the scalp has a frequency in the range 6000-12000 nm and can be received remotely without physical contact. The amount of IR reflected by the gemstones depends on the total surface area of a gemstone powder particle and the number of gemstone particles. Thus, it is better to have gemstone particles that are as small as possible. Gemstone powder particles could be set to pass through mesh #20 (around 0.84 mm or smaller).

The inventors believe that the energy emitted from the gemstones powder may act as a form of LLLT to restore and balance the distorted energy flow in the twelve meridian networks and the seven chakras in order to activate and enhance the self-recovery of brain degeneration that cause disorders such as dementia and Parkinson's disease.

Each module includes at least one output component and other control components that control the output component or components.

Referring now to FIGS. 4A-4D, a CDD 10 may include a circuit board 48. Circuit board 48 carries at least the microcontroller unit (MCU) 12, and other control components that enable free electron generation by free electron generator module 14, IR by the infrared generator (IR generator) module 16, heat by the heater module 18, and visible light by the visible light generator module 20. When present, the regulator 24 may also be located on the circuit board 48. It should be noted that while the embodiment disclosed herein uses the circuit board 48 for carrying the electronic components that enable the functioning of the modules, it is possible an application specific integrated circuit could be devised to perform the same functions as the components on the circuit board 48.

The circuit board 48 is mounted (with screws, for example) on the rear face of a battery carrier 50, in which the battery 22 is received. A removable cover 51, which closes an opening in the main body 36 to prevent access to the battery 22, provides access to the battery 22 when removed. Battery terminals 53 extend from the rear side of the circuit board 48 and into the battery carrier 50 in order to make contact with the positive and the negative poles of the battery 22 as is well known.

Two heater elements 52 are the output components of the heater module 18. Each heater element 52 is received in a respective stud 32, 34 in order to heat the stud 32, 34 when the heater module 18 is operating. The heater elements 52 may heat the studs to a temperature in the range 35°-40° C., for skin contact applications, and 45°-65° C., for non-skin contact applications.

Furthermore, as mentioned above, both studs 32, 34 are output components of the free electron generator module 14, one serving as the charge head electrode 14′ and the other serving as the discharge head 14″ electrode. It does not matter which one serves as the charge head 14′ electrode or the discharge head 14″ electrode.

In addition, each stud 32, 34 is a receptacle for, and has deposited therein, gemstones in powder form. The gemstones powder may be the same combination of gemstones in powder as those in the container 45. The purpose of the gemstones in powder form is to absorb the IR emitted by the brain continuously, and upon receiving sufficient energy level from the IR emitted by the brain, the gemstone powder will emit its own IR to bring about the effects of LLLT. The gemstones absorb IR emitted from the human body continuously.

An LED part 54, which is the output component of the visible light generator module 20, is arranged to face the bottom of the head section 30 aligned with an orifice 56 defined in the bottom of the head section 30 to allow the transmission of visible light. The LED part 54 may be a component that emits a light at one frequency (e.g. red (650 nm), green (450 nm, blue (380 nm)) or may be a component that emits multiple frequencies of light (e.g. red (650 nm), green (450 nm, blue (380 nm)). Orifice 56 is preferably located between openings 58 defined in the head section 30. Each stud 32, 34 extends through a respective opening 58.

An IR generating coil 60 is the output component of the IR generator module 16. The IR generating coil 60 is arranged around the studs 32, 34. The IR generating coil 60 may be a far IR (FIR) coil. FIR refers to a specific wavelength range (6000-12000 nm) within the IR spectrum.

The IR generating coil 60 and the LED part 54 may be cyclically operated (turned on and off) with a DC input at various frequencies (i.e. duty cycles). A preferred frequency is 7.83 Hz, which is known as the Schumann Resonance. Stimulus delivered at the Schumann Resonance can alleviate insomnia, which is mostly caused by toxin accumulation (including hormones like cortisol inside the brain). The IR generating coil 60 and the LED component 54 may be operated at other harmonic frequencies, including 14.07 Hz, 20.25 Hz, 26.41 Hz, and 32.45 Hz to address the cause(s) of dementia. The operation frequency range of the IR generating coil 60 and the LED part 54 may be 5-50 Hz. The exact frequency can be set by the user, for example, through an app on a mobile device that is wirelessly connected to a CDD according to the present invention. It should be noted that the head section 30 is made of a material that does not hinder the transmission of IR from the IR generating coil 60. That is, the head section 30 does not absorb the IR radiated from the IR generating coil 60 and is thus transparent to the IR radiated from the IR generating coil 60.

In one mode, power at a designated operating frequency may be supplied to the IR generating coil 60 and the LED part 54 simultaneously and at the same frequency so that the visible light from the LED part 54 and the FIR from the IR generating coil 60 stimulate the skin of the cranial skull and the acupressure points allocated on the cranial skull together.

The LED part 54 may be located on its own board 62. A support 64 is provided to support the studs, 32, 34, the board 62, the IR generating coil 60, and the heating elements 52.

For the sake of clarity, the control components of the modules are not shown in the figures. It is submitted that the control components that control the output component(s) of each module and their manner of connection to the output component(s) are known and need not be described in detail.

In one embodiment, an app residing on a mobile device such as a smart phone may be used to control/adjust the output of the modules including the heat output, the frequency, the light photon intensity, and the electrical power output.

In addition, the main body 36 may be a multiple part component or a may be a single, unitary piece.

Preferably, except for the heating elements 52, which extend from the interior of the main body 36 to the interior of the head section 30, the other output components of the modules are inside of the head section 30.

In addition, preferably each stud 32, 34 has a dome-shaped end at the end of a cylindrical body. The dome-shaped ends are configured to engage the human scalp without causing great discomfort.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A cranial detoxifier device (CDD), comprising:
a free electron generator module;
an infrared (IR) generator module;
a heater module;
a visible light generator; and
a microcontroller that controls the free electron generator module, the infrared generator module, the heater module, and the visible light generator.

2. The CDD of claim 1, further comprising a housing that houses the microcontroller and components of the free electron generator module, the IR generator module, the heater module, and the visible light generator.

3. The CDD of claim 2, further comprising a first metallic stud and a second metallic stud extending out of a head section of the housing, each metallic stud having a dome-shaped end.

4. The CDD of claim 2, further comprising a first metallic stud and a second metallic stud extending out of a head section of the housing, the first and second metallic studs serving as output components of the free electron generator module, and transmit heat from the output components of the heater module.

5. The CDD of claim 2, further comprising a first metallic stud and a second metallic stud extending out of a head section of the housing, each metallic stud containing gemstones in powder form.

6. The CDD of claim 2, further comprising an LED component serving as an output component of the visible light generator, and an orifice defined in the head section, wherein the LED component is aligned with the orifice to transmit light through the head section.

7. The CDD of claim 2, further comprising a first metallic stud and a second metallic stud extending out of a head section of the housing, the first and second metallic studs serving to transmit heat from the output components of the heater module in the temperature range of 35°-65° C.

8. The CDD of claim 2, further comprising an infrared generating coil serving as an output component of the IR generator module residing within a head section of the housing.

9. The CDD of claim 2, further comprising an infrared (IR) generating coil serving as an output component of the IR generator module residing within a head section of the housing, and an LED component serving as an output component of the visible light generator residing in the head section, the IR generating coil and the LED component being operable in the range 5-50 Hz.

10. The CDD of claim 9, wherein the LED component emits one of red light, green light, and blue light.

11. The CDD of claim 9, wherein the LED component emits red light, green light, and blue light.

12. The CDD of claim 2, further comprising a first metallic stud and a second metallic stud extending out of a head section of the housing, the first and second metallic studs serving as a charge head and a discharge head of the free electron generator module, wherein, in operation, the charge head is at a voltage in the range 20-30 volts relative to the discharge head and the free electron generator module generates less than 0.03 amps.

13. The CDD of claim 2, further comprising a container containing gemstones in powder form.

14. The CDD of claim 13, wherein the gemstones include pure and clear white quartz, amethyst, clear aquamarine, rose quartz, clear citrine, Tiger's Eye, and Tektite.

15. The CDD of claim 14, wherein particles of the gemstones in the powder form are 0.84 mm or smaller.

16. The CDD of claim 5, wherein the gemstones include pure and clear white quartz, amethyst, clear aquamarine, rose quartz, clear citrine, Tiger's Eye, black obsidian and Tektite.

17. The CDD of claim 16, wherein particles of the gemstones in the powder form are 0.84 mm or smaller.

18. The CDD of claim 1, wherein output of the free electron generator module, the IR generator module, the heater module, and the visible light generator are adjustable in response to user instructions.

19. The CDD of claim 18, wherein user instructions are received from a user-operable mobile device.

20. A system comprising two or more CDDs according to claim 2, and a carrier, wherein each CDD has first and second metallic studs extending from a head section of the housing thereof, and wherein each housing of each CDD has exterior threads that engage a threaded inner surface of a respective opening in the carrier whereby rotation of each CDD housing causes repositioning of the first and second metallic studs thereof relative to the carrier.
